SEQUENCE LISTING (4 sequences)

SEQ ID NO: 1 - protein, 274 aa, Artificial Sequence
  potato StWhy1 protein sequence
MSNFSLSPSPTSGFSLNLQNPTKTSYLSFSSSINTIFAPLSSNTTKSFSGLTHKAALPRNLSLTCRHSDYFEPQQQQQQQQQQPQGASTPKVFVGYSIYKGKAALTVEPRSPEFSPLDSGAFKLSREGMVMLQFAPAAGVRQYDWSRKQVFSLSVTEIGSIISLGAKDSCEFFHDPNKGRSDEGRVRKVLKVEPLPDGSGHFFNLSVQNKLINLDENIYIPVTKAEFAVLVSAFNFVMPYLLGWHTAVNSFKPEDASRSNNANPRSGAELEWNR

SEQ ID NO: 2 - protein, 263 aa, Artificial Sequence
  Arabidopsis Whirly proteins AtWhy1
MSQLLSTPLMAVNSNPRFLSSSSVLVTGGFAVKRHGFALKPTTKTVKLFSVKSRQTDYFEKQRFGDSSSSPSPAEGLPARFYVGHSIYKGKAALTVDPRAPEFVALDSGAFKLSKDGFLLLQFAPSAGVRQYDWSKKQVFSLSVTEIGTLVSLGPRESCEFFHDPFKGKSDEGKVRKVLKVEPLPDGSGHFFNLSVQNKLVNVDESIYIPITRAEFAVLISAFNFVLPYLIGWHAFANSIKPEETSRVNNASPNYGGDYEWNR

SEQ ID NO: 3 - protein, 237 aa, Artificial Sequence
  Arabidopsis Whirly proteins AtWhy2
MKQARSLLSRSLCDQSKSLFEASTLRGFASWSNSSTPGRGFPGKDAAKPSGRLFAPYSIFKGKAALSVEPVLPSFTEIDSGNLRIDRRGSLMMTFMPAIGERKYDWEKKQKFALSPTEVGSLISMGSKDSSEFFHDPSMKSSNAGQVRKSLSVKPHADGSGYFISLSVNNSILKTNDYFVVPVTKAEFAVMKTAFSFALPHIMGWNRLTGHVNTEALPSRNVSHLKTEPQLELEWDK

SEQ ID NO: 4 - protein, 267 aa, Artificial Sequence
  Arabidopsis Whirly proteins AtWhy3
MSQLLSSPPMAVFSKTFINHKFSDARFLSSHSILTSGGFAGKIIPLKPTARLKLTVKSRQSDYFEKQRFGDSSSSQNAEVSSPRFYVGHSIYKGKAALTIEPRAPEFVALESGAFKLTKEGFLLLQFAPAAGVRQYDWSRKQVFSLSVTEIGNLVSLGPRESCEFFHDPFKGKGDEGKVRKVLKVEPLPDGSGRFFNLSVQNKLLNVDESVYIPITKAEFAVLISAFNFVLPHLIGWSAFANSIKPEDSNRLNNASPKYGGDYEWSR

(19) United States
(12) Patent Application Publication
(10) Pub. No.: US 2007/0074306 A1
(43) Pub. Date: Mar. 29, 2007

(54) Plant transcriptional activator and uses thereof

(76) Inventors: Normand Brisson, Quebec (CA); Darrell Desveaux, Ontario (CA); Rajagopal Subramaniam, Ontario (CA); Jurgen Sygusch, Quebec (CA)

Correspondence Address: CHOATE, HALL & STEWART LLP, TWO INTERNATIONAL PLACE, BOSTON, MA, 02110, US

(21) Appl. No.: 10/561,053

(22) PCT Filed: Jun. 18, 2004
(86) PCT No.: PCT/CA04/00907

§ 371 Date: Jun. 26, 2006

Related U.S. Application Data
(60) Provisional application No. 60/479,871, filed on Jun. 20, 2003.

(51) Int. Cl. A01H 1/00; C07K 14/415; C07H 21/04; C12N 15/82; C12N 5/04

(52) U.S. Cl. 800/279; 435/419; 435/468; 530/370; 536/23.6

(57) ABSTRACT

The present invention relates to plant transcriptional activators and mutants thereof. Furthermore, the present invention relates to uses of plant transcriptional activators and mutants thereof for increasing plant defence responses to pathogens. In particular, the present invention relates to a peptide which confers increased pathogen resistance upon a plant expressing said peptide, said peptide having a sequence selected from the group consisting of SEQ ID NO:1, SEQ ID NO:2, SEQ ID NO:3, and SEQ ID NO:4 mutated at a position selected from the group consisting of Glu271, Trp272 and residues between and including Pro123 to Gly128, an ortholog thereof, a homolog thereof, a functionally active fragment thereof or a functionally active variant thereof.

### BACKGROUND OF THE INVENTION

(a) Field of the Invention

The present invention relates to plant transcriptional activators and mutants thereof, Furthermore, the present invention relates to uses of plant transcriptional activators and mutants thereof for increasing plant defence responses to pathogens.

(b) Description of Prior Art

A variety of defence specific events are induced in plants in response to pathogen infection. Although key components of the signaling cascades are being discovered, few transcription factors that integrate these signals at the transcriptional level have been identified to date.

PR genes are plant genes that are induced by pathogen invasion. These genes are subdivided into 11 classes. Since PR genes are well characterized, they provide excellent models to study transcriptional regulation of defence genes.

The PR-10 gene family is one of the classes of PR genes. Expression studies have identified cis-acting elements involved in PR-10a gene regulation, a member of the PR-10 gene family (Matton et al. 1993, Plant Mol. Biol. 22:279-291). An elicitor response element (ERE) located between nucleotides −135 and −105 is essential and sufficient for elicitor-induced expression of PR-10a (Després et al. 1995, Plant Cell 7:589-598). PBF-2, a single-stranded DNA binding factor, appears to play a role in activation of PR-10a from the ERE (Desveaux et al. 2000, Plant Cell 12:1477-1489). It has been shown that the presence of the ERE is sufficient for PR-10a activation. It has also been shown that the sequence that is bound by PBF-2 is GTCAAAAA. It has been shown that, in planta, PBF-2 binds to PR-10a only when this gene is activated by wounding or by treatment with an elicitor that mimics the action of a pathogen. PBF-2 is a tetramer made of four identical 24 kD (p24) subunits (Desveaux et al. 2002, Nature Struct. Biol. 9:512-517). The sequence and secondary structure of p24 is conserved among plant species and this novel plant transcription factor has been renamed Whirly, based on the whirligig appearance of the quaternary structure of the protein. Accordingly the potato p24 has been renamed StWhy1, and its ortholog in Arabidopsis AtWhy1.

It would be highly desirable to be provided with plant transcriptional activators, mutants thereof and uses thereof for increasing plant defence responses to pathogens.

### SUMMARY OF THE INVENTION

One aim of the present invention is to provide plant transcriptional activators and mutants thereof.

Another aim of the present invention is to provide plant transcriptional activators and mutants thereof for increasing plant defence responses to pathogens.

In accordance with the present invention there is provided plant transcriptional activators and mutants thereof.

In accordance with the present invention there is also provided plant transcriptional activators and mutants thereof for increasing plant defence responses to pathogens.

In accordance with one embodiment of the present invention there is provided a peptide which confers increased pathogen resistance upon a plant expressing the peptide, the peptide having a sequence selected from the group consisting of SEQ ID NO:1, SEQ ID NO:2, SEQ ID NO:3, and SEQ ID NO:4 mutated at a position selected from the group consisting of Gly148, Pro183, Glu271, Trp272 and residues between and including Pro123 to Gly128, an ortholog thereof, a homolog thereof, a functionally active fragment thereof or a functionally active variant thereof.

In accordance with another embodiment of the present invention there is provided a recombinant nucleic acid molecule comprising a sequence which codes for a peptide selected from the group consisting of SEQ ID NO: 1, SEQ ID NO:2, SEQ ID NO:3 and SEQ ID NO:4 mutated at a position selected from the group consisting of Gly148, Pro183, Glu271, Trp272 and residues between and including Pro123 to Gly128, an ortholog thereof, a homolog thereof, a functionally active fragment thereof or a functionally active variant thereof.

A preferred recombinant nucleic acid molecule is DNA.

Preferably a vector contains the recombinant nucleic acid molecule, more preferably an expression vector.

A preferred recombinant nucleic acid molecule is operatively linked to an expression control sequence.

In accordance with another embodiment of the present invention there is provided a method of expressing a recombinant nucleic acid molecule in a cell containing an expression vector of the present invention, comprising culturing the cell in an appropriate cell culture medium under conditions that provide for expression of the recombinant DNA molecule by the cell.

A preferred method of the present invention, further comprises the step of purifying a recombinant product of the expression of the recombinant DNA molecule.

In accordance with another embodiment of the present invention there is provided a cell transformed with the recombinant DNA molecule of the present invention.

In a preferred embodiment of the present invention, the recombinant DNA molecule is integrated in the genome of the cell.

A preferred cell of the present invention is a plant cell.

In accordance with another embodiment of the present invention there is provided a cell expressing the peptide of the present invention, a plant comprising such cell.

In accordance with another embodiment of the present invention there is provided a transgenic plant either expressing the peptide of the present invention or comprising a recombinant nucleic acid molecule of the present invention.

In a preferred transgenic plant of the present invention the recombinant nucleic acid is integrated into the genome of the cell.

In accordance with another embodiment of the present invention there is provided a method of increasing pathogen resistance in a plant comprising the steps of: (a) introducing into a cell of the plant a recombinant nucleic acid molecule of the present invention; and (b) expressing the recombinant nucleic acid molecule in the cell.

In accordance with another embodiment of the present invention there is provided a method of increasing pathogen resistance in a plant comprising the steps of: (a) mutating a nucleic acid sequence which codes for p24; and (b) expressing the nucleic acid sequence in the plant, wherein the mutating results in a amino acid substitution in the p24 which increases DNA binding affinity of PBF-2 for an elicitor response element (ERE).

In a preferred method of the present invention the amino acid substitution replaces Pro125 with nothing or a different amino acid, preferably with Leu.

In a preferred method of the present invention the amino acid substitution replaces Trp272 with nothing or a different amino acid, preferably with Ala.

In a preferred, method of the present invention the amino acid substitution replaces Glu271 with nothing or a different amino acid, preferably with any non-acidic amino acid.

In a preferred method of the present invention the amino acid substitution replaces Pro183 with nothing or a different amino acid, preferably with Ser.

In a preferred method of the present invention the amino acid substitution replaces Gly148 with nothing or a different amino acid, preferably with Glu.

In a preferred method of the present invention the ERE regulates expression of a pathogenesis-related (PR) gene.

In a preferred method of the present invention the PR gene is a PR-10 gene, preferably PR-10a.

In a preferred method of the present invention the step of mutating a nucleic acid sequence is effected by a chemical mutagen, radiation, natural mutation or a recombinant DNA technique, preferably site-directed mutagenesis.

In accordance with another embodiment of the present invention there is provided a method of increasing pathogen resistance in a plant comprising increasing DNA binding affinity of PBF-2 for an elicitor response element (ERE) of a pathogenesis-related (PR) gene.

In a preferred method of the present invention increasing DNA binding affinity of PBF-2 for an ERE comprises mutating a C-terminal negative autoregulatory domain of p24, wherein the C-terminal autoregulatory domain inhibits PBF-2 DNA binding and wherein the mutating decreases negative autoregulation of the domain.

In a preferred method of the present invention the mutating comprises an amino acid substitution in p24.

In a preferred method of the present invention mutating a C-terminal negative autoregulatory domain is effected by a chemical mutagen, radiation, natural mutation or a recombinant DNA technique, preferably site-directed mutagenesis.

In a preferred method of the present invention the amino acid substitution replaces a residue between and including Pro123 to Gly128 with nothing or a different amino acid.

For the purpose of the present invention the following terms are defined below.

The term “ortholog” is intended to mean a gene obtained from one species that is structurally similar and is the functional counterpart of a gene from a different species. Sequence differences among orthologs are the result of speciation.

The term “homolog” is intended to mean a gene or protein from one species, that has a common origin and functions the same as a gene or protein, respectively, from another species.

The term “transformed” when qualifying a cell is intended to mean a cell into which (or into an ancestor of which) has been introduced, by means of recombinant DNA techniques, a DNA molecule encoding (as used herein) AtWhy1, a homolog of AtWhy1, a functional mutant of AtWhy1, a functional fragment of AtWhy1, a functional fragment of a homolog of AtWhy1, and a functional fragment of a functional mutant of AtWhy1.

The term “transgenic” is intended to mean an organism harbouring in its genome of its germ and/or somatic cells a transgene that has been introduced using recombinant technology.

The term “transgene” is intended to mean a gene inserted into the genome of the germ and/or somatic cells of an organism in a manner that ensures its function, replication and transmission as a normal gene. A “transgene” can be any piece of a nucleic acid molecule (for example, DNA) which is inserted by artifice into a cell, and becomes part of the organism (integrated into the genome or maintained extrachromosomally) which develops from that cell. Such a transgene may include a gene which is partly or entirely heterologous (i.e., foreign) to the transgenic organism, or may represent a gene homologous or analogous to an endogenous gene of the organism.

The term “pathogen” is intended to mean any organism which can infect another organism. Such infection may result in and/or induce a disease in the infected organism and/or result in the death of the infected organism. Examples of pathogens include, but are not limited to, bacteria, viruses, fungi, oomycetes, insects, nematodes and plants.

### BRIEF DESCRIPTION OF THE DRAWINGS

FIG. 1 illustrates the sequence alignment, of the potato StWhy1 protein sequence (SEQ ID NO:1) and the three Arabidopsis Whirly proteins AtWhy1 (gi5223748) (SEQ ID NO:2), AtWhy2 (gi18175814) (SEQ ID NO:3) and AtWhy3 (gi15227028) (SEQ ID NO:4);

FIG. 2 illustrates that mutations in Arabidopsis AtWhy1 lead to change in the susceptibility of Arabidopsis thaliana cotyledons to infection by Peronospora parasitica Noco2, as quantified by counting sporangia on cotyledons 7 days after infection;

FIG. 3 illustrates wild type Col-0 Arabidopsis plants and AtWhy1 TILLING lines till1, till2 and till3 infected with the compatible oomycete pathogen P. parasitica isolate Noco2;

FIG. 4 illustrates that the extreme C-terminal domain of StWhy1 interacts with and appears to interfere with the DNA-binding surface of StWhy1 through an interaction mediated mainly through Lys188 and Trp272, as well as Glu271 and His174;

FIG. 5 illustrates that mutation of Trp272 increases the DNA-binding affinity of StWhy1 and shows (A) the mutation mW272 causing a change in electrophoretic mobility relative to wild type StWhy1, (B) a bar graph quantitatively representing the amount of probe shifted by each protein in (A) relative to wild type StWhy1 (StWhy1=1.0), and (C) immunoblot analysis of extracts used for EMSA analysis in (A) using antibody raised against recombinant StWhy1 to show equal loading of the Wt and m272 proteins;

FIG. 6 illustrates a western blot showing that the overexpression of AtWhy1-TAP fusion protein leads to an overexpression of the SAR marker PR-1

FIG. 7 illustrates the intracellular localization of p24 in potato leaves;

FIG. 8 illustrates the intracellular localization of p24-GFP in roots;

FIG. 9 illustrates the chloroplast localization of p24-GFP in mesophyll cells;

FIG. 10 illustrates the binding of p24 to YCF3 on chloroplast DNA;

FIG. 11 illustrates the Ycf3 RNA increase in wounded p30 potato; and

FIG. 12 illustrates the PSI-D Protein Level increase in wounded p30-potato.

### DETAILED DESCRIPTION OF THE INVENTION

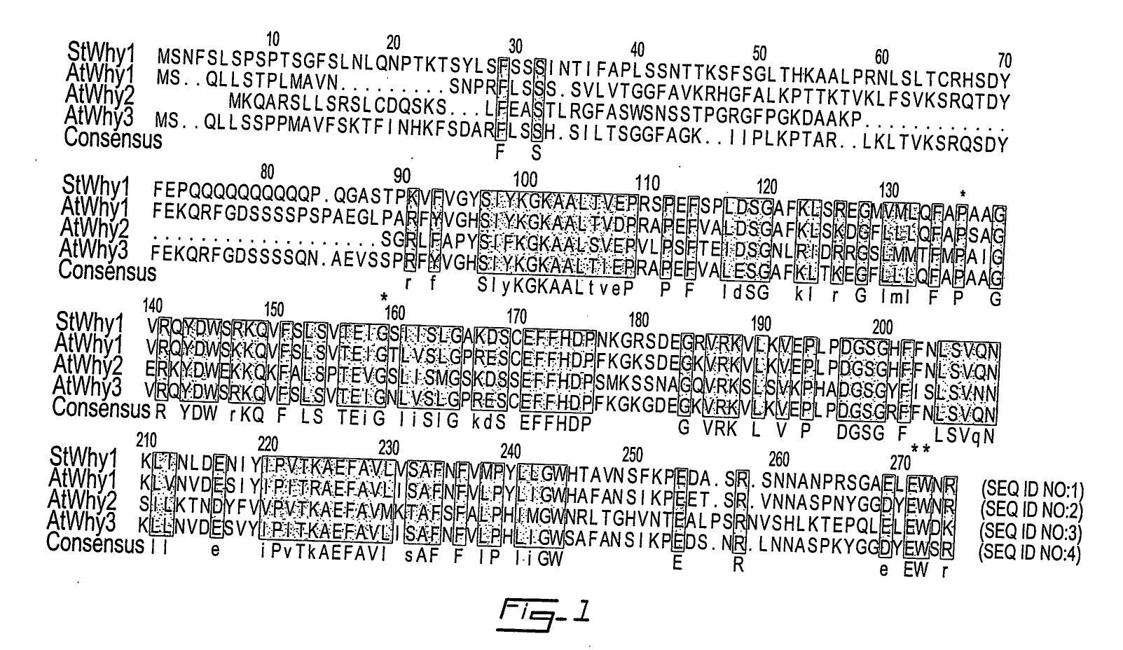

Sequence alignment of the potato StWhy1 protein sequence (SEQ ID NO:1) and the three Arabidopsis Whirly proteins AtWhy1 (gi5223748) (SEQ ID NO:2), AtWhy2 (gi18175814) (SEQ ID NO:3) and AtWhy3 (gi15227028) (SEQ ID NO:4) is shown in FIG. 1. The alignment was initially performed using ClustalW (Thompson et al. 1994, Nucl. Acids Res. 22:4673-4680) and was subsequently manually modified. Numbering corresponds to the StWhy1 protein. Conserved residues (100% between all four members) are boxed in black and positions with conserved similar residues are boxed in grey. The consensus protein sequence obtained from the alignment is indicated at the bottom.

The secondary structure predicted by the program PDH indicates that these sequences are likely to adopt a secondary structure similar to that of potato p24 (Desveaux et al. 2002, supra).

Arabidopsis lines containing single point mutations in the sequence of AtWhy1 were obtained from the Arabidopsis Tilling Project (McCallum et al. 2000, Nature Biotechnol. 18:455-457). Three Arabidopsis lines, till1,till2 and till3 (till2 and till 3 may also be referred to herein as AtWhy1.1 and AtWhy1.2, respectively) possessing point mutations in AtWhy1 were obtained by TILLING. The mutation in line till1 changes Pro125 to Leu, till2 changes Pro183 to a Ser and the mutation in till3 changes Gly148 to a Glu. The mutations in the Arabidopsis TILLING lines are marked by an asterisk in FIG. 1, as well as the amino acids Trp272 and Glu271 believed to negatively regulate DNA-binding activity. The TILLING lines were produced by treatment of Arabidopsis seeds with a mutagenizing agent.

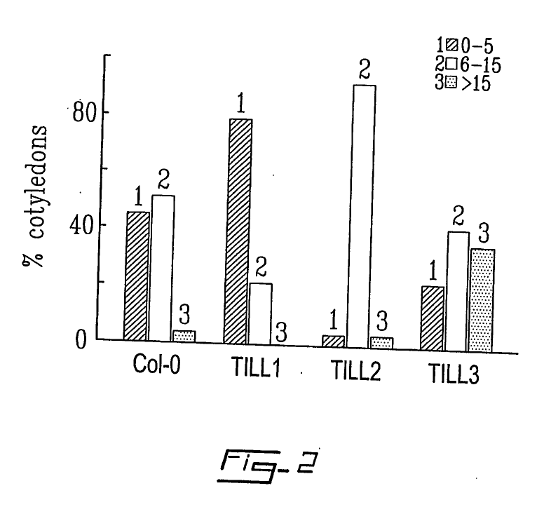

FIG. 2 shows the susceptibility to infection by Peronospora parasitica isolate Noco2 of wild type Arabidopsis thaliana Col-0, of two mutant lines that show increased susceptibility to this pathogen (lines till2 and till3), and of the (Pro125 to Leu) mutant line (till1), which shows decreased susceptibility to infection. Mutations in Arabidopsis AtWhy1 that lead to change in the susceptibility to infection by Peronospora parasitica isolate Noco2 were quantified by counting sporangia on cotyledons 7 days after infection. Cotyledons were scored as either having 0-5,6-15 or greater than 15 sporangia. Bars of the histogram represent the percentage of cotyledons falling into the three categories of sporangia counts for each genotype infected. Each category is represented by a different bar code: bar 1, 0-5 sporangia; bar 2, 6-15 sporangia; bar 3, more than 15 sporangia per cotyledon.

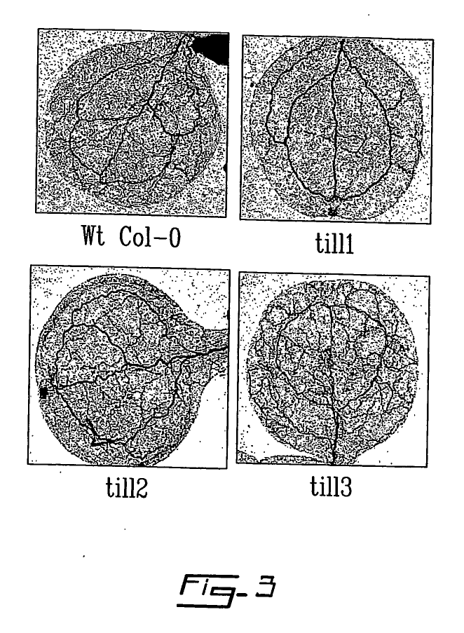

These results are confirmed by staining infected cotyledons with Trypan Blue. Wild type Col-0 Arabidopsis plants and AtWhy1 TILLING lines till1, till2 and till3 were infected with the compatible oomycete pathogen P. parasitica isolate Noco2. Hyphal growth was then examined by Trypan Blue staining leaves 2 days after infection (FIG. 3).

Replacement of Pro125 with amino acids other than leucine and the modification of amino acid residues surrounding Pro125 also lead to increased resistance to pathogens.

The above results suggest that the mutation Pro125 to Leu in AtWhy1 is dominant. Overexpression of this mutant AtWhy1 gene in Arabidopsis thaliana also confers a disease resistant phenotype. Transformation of any plants with the AtWhy1 mutant gene bearing the Pro125 to Leu mutation, or an homolog of AtWhy1 with a similar mutation, should confer to this plant an increased resistance to pathogens.

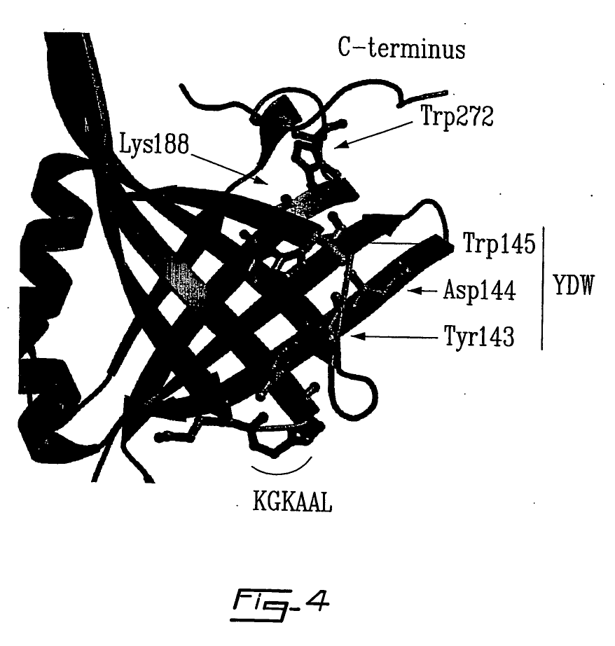

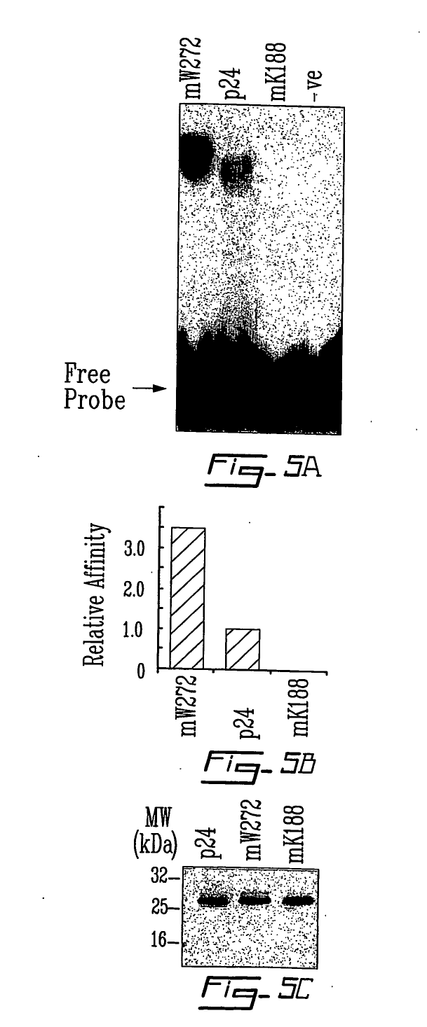

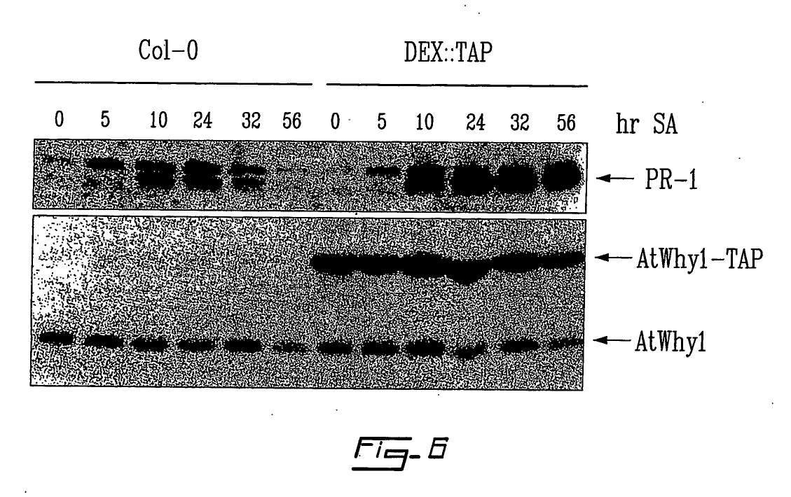

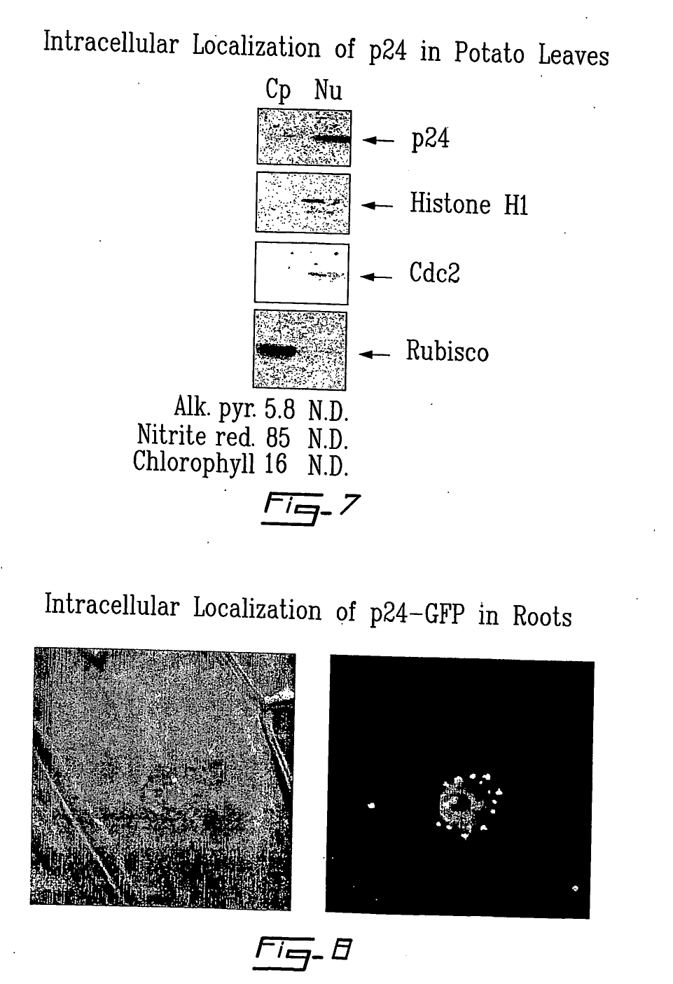

Sequence comparison of Whirly family members reveals that Lys188 is conserved among these proteins (Desveaux et al, 2002, supra: FIG. 1). Furthermore, the side chain of this residue is exposed to the solvent and its position suggests that it could make contact with single-stranded DNA (ssDNA) (FIG. 4). Interestingly, the crystal structure of PBF-2 revealed that this residue interacts with Trp272 located at the C-terminus (FIG. 4). This interaction positions the C-terminus across the β-sheet surface of PBF-2, where it could interfere with DNA binding. As shown in FIG. 4 the C-terminal domain of StWhy1 interacts with and interferes with the DNA-binding surface of StWhy1 through an interaction mediated mainly through Lys188 and Trp272, as well as Glu271 and His174. The DNA binding surface of a PBF-2 protomer is depicted as a ribbon diagram in FIG. 4 with the side chains of conserved amino acid residues predicted to be important for ssDNA binding affinity indicated. Amino acids of the KGKAAL sequence known to be critical for DNA binding activity are located at positions 100-105, those of the YDW sequence in loop L₃₄ are at positions 143-145, the C-terminal backbone containing Trp272 is labelled as C-terminus, and the position of Lys188 is indicated by an arrow.

Mutation of Lys188 abolished PBF-2 DNA binding activity in Electro Mobility Shift Assays (EMSA), confirming the importance of this residue for DNA binding. Therefore, the C-terminus not only acts as a barrier to the ssDNA, but competes with the DNA for interaction with Lys188.

As shown in FIG. 5, mutation of Trp272 to Ala in the C-terminus of StWhy1 (FIG. 1; mW272) resulted in a 3.5-fold increase in DNA binding affinity, indicating that the C-terminal region of p24 acts as a negative autoregulatory domain. Trp272, which appears to be important for interacting with the DNA-binding surface of StWhy1 was mutated and its effect on DNA binding activity was examined. FIG. 5A shows the EMSA analysis of wild type StWhy1 and the mutations mW272 and mK188 with the non-coding strand of the ERE as probe using 80 nM of protein in each reaction. Note that the mutation mW272 causes a change in electrophoretic mobility relative to wild type StWhy1, suggesting a possible important conformational change. FIG. 5B shows a bar graph quantitatively representing the amount of probe shifted by each protein in FIG. 5A relative to wild type StWhy1 (StWhy1=1.0). Quantitation of DNA bound by StWhy1 was assessed by liquid scintillation counting of the retarded band excised from the gel. FIG. 5C shows an immunoblot analysis of extracts used for EMSA analysis in FIG. 5A using antibody raised against recombinant StWhy1 to show equal loading of the Wt and mW272 proteins. It is understood that the majority of amino acids, particularly those similar to Ala, would give similar results.

Therefore, mutation of Trp272 which leads to increase binding of PBF-2 to DNA leads to increased resistance to pathogens. This is supported by the observation that the AtWhy1 mutant protein in the till2 and till3 lines (more susceptible to infection, FIGS. 2 and 3) bind significantly less to DNA than wild type AtWhy1. This indicates that a correlation exists between the extent of binding to DNA and disease resistance. The Trp272 mutation could be obtained through mutagenesis of the wild type gene in planta. Alternatively plants could be rendered more resistant by transformation with the Trp272 mutant allele, using such techniques as, but not limited to, Agrobacterium meditated transformation, particle bombardment, direct DNA transformation, viral vector infection, electroporation, and micro-injection.

Examination of the crystal structure of PBF2 revealed that a second amino acid, Glu271, also contributes to the interaction of the C-terminus across the β-sheet surface of PBF-2. It is therefore anticipated that mutation of this residue also leads to increased DNA binding, and therefore to an increased resistance to pathogens.

The present invention also shows that another point mutation (Pro125 to Leu) in the sequence of AtWhy1 (till1) confers to the plant Arabidopsis thaliana an increased resistance to infection by the pathogen Peronospora parasitica isolate Noco2 (FIGS. 2 and 3). These figures show that less sporangia and hyphal growth are present in till1 plants infected with the oomycete as compared to wild type plants infected with the same pathogen. By contrast, point mutations in the till2 and till3 lines lead to increase susceptibility to infection by P. parasitica, confirming the importance of AtWhy1 in disease resistance.

Furthermore, the DNA binding activity of this protein has now been shown to be induced by treatment with salicylic acid (SA), an inducer of disease resistance, and also by the incompatible pathogen Peronospora parasitica Emoy2. SA induces AtWhy1 DNA binding activity independently of the regulator NPR1. It has also been shown that AtWhy1 is required for the establishment of SA-induced disease resistance.

These results indicate that it is possible to screen for the presence of mutations leading to increased disease resistance in Whirly genes in any plant species and that these mutations confer increased resistance to infection by pathogens. These mutations could be induced by any means, including but not limited to, chemical, radiation, natural, or alternatively by using recombinant DNA techniques such as site-directed mutagenesis on an isolated nucleic acid sequence.

Similar mutations in other Whirly gene family members are also expected to lead to resistance to infection.

Increased resistance to infection by pathogens can also be effected by overexpressing AtWhy1, StWhy1, or any peptide of the present invention or an ortholog thereof, or an analog thereof that binds to the PR gene, and more particularly to the PR-1 or PR-10 gene, and more preferably the PR-10a. In Fact, in FIG. 6, it is shown that DEX::TAP transgenic plants overexpress the AtWhy1-TAP fusion protein following dexamethazone (DEX) treatment.

In FIG. 6, the AtWhy1 gene (WT) was fused to a tag sequence (TAP-tag) and placed under the control of a dexamethasone (DEX) inducible promoter. Following transformation of Arabidopsis, plants were treated with dexamethasone for 48 h and then the plants were sprayed with salicylic acid (SA), an inducer of the defense response against pathogens, and total proteins were extracted 0, 5, 10, 24, 32, 56 hours post SA treatment. Western blot analysis was conducted with PR-1 and AtWhy1 specific antibodies. The accumulation of the PR protein PR-1, a marker of the defense response, was then monitored by immunoblotting.

The results show that upon treatment of wild type (Col-0, untransformed) plants with SA, PR-1 starts accumulating after 10 hrs and declines after 32 hrs. The plants overexpressing AtWhy1 (DEX::TAP) show a higher accumulation of PR-1, and its level remains high even after 56 hrs of treatment with SA. This is strongly indicative of a higher induction of the defense response in AtWhy1 overexpressing plants.

In FIG. 6, the upper panel was probed with an anti-PR-1 antibody; the lower band is PR-1. The lower panel was probed with an anti-AtWhy1 antibody; the lower band represents the endogenous AtWhy1 and shows equal loading.

The gene encoding AtWhy1 and its potato homolog, StWhy1, encodes a 30 kD precursor protein (p30) containing a transit sequence. It is therefore predicted that these proteins could be present in plastids, such as the chloroplast. The intracellular localization of StWhy1 was directly determined by immunoblot. FIG. 7 reveals that the protein is present in both the chloroplast and the nuclear fraction. This was confirmed by expressing a fusion consisting of the Green Fluorescence Protein (GFP) fused to potato p30 (StWhy1) in transgenic tobacco. In FIG. 7, nuclear and chloroplast protein fractions were isolated from potato leaves and analysed by immunoblotting after gel electrophoresis. Anti-p24 antibody reveals that the protein is present in both the chloroplast and the nucleus (p24). The nuclear protein markers histone H1 and Cdc2 indicates that the chloroplast fraction is not significantly contaminated with nuclear proteins. The chloroplast markers rubisco and chlorophyll, as well as the enzymatic activities of the chloroplast enzymes alkaline pyrophosphatase and nitrite reductase indicate that the nuclear fraction is not significantly contaminated by chloroplast proteins. In FIG. 7, Cp refers to chloroplast; Nu refers to nucleus and N.D. means not detectable.

Analysis of transformed tobacco tissues confirmed that the protein is present in the nucleus and the chloroplast (FIG. 8). In the chloroplast, the protein is localized in speckles that co-localize with DNA, suggesting that p24 is bounded to DNA. This was confirmed by immunoprecipitation of the protein that had been cross-linked to DNA. In FIG. 8, a root section of a tobacco plant expressing the fusion protein p24-GFP was examined by confocal microscopy. The left panel shows the phase contrast image of a nucleus in a root cell. The right panel shows the green fluorescence image of the same cell. Fluorescence is detected in both the nucleus and the small surrounding plastids.

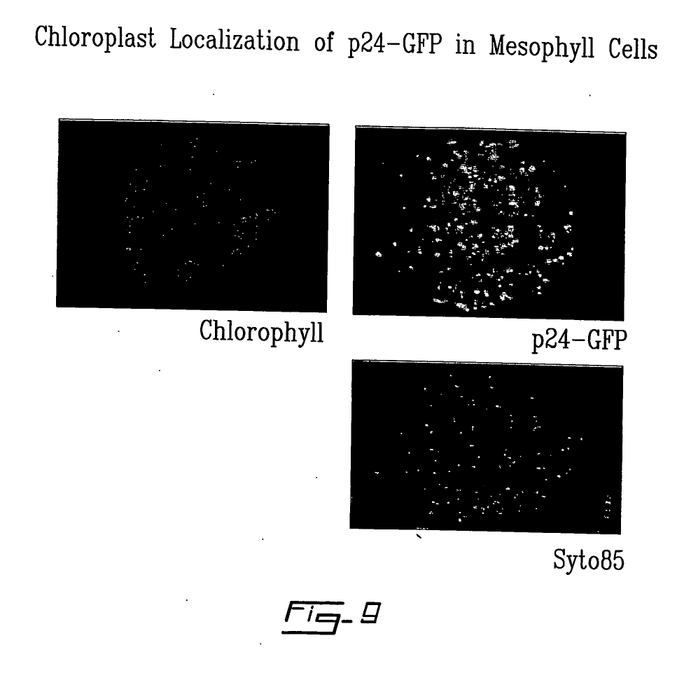

The protein is associated to a small region of the YCF3 gene containing the PB regulatory element, suggesting that it may play a role in the transcriptional regulation of this plastid gene (FIG. 9). In FIG. 9, a tobacco leaf mesophyll cell was transfected with a vector encoding a p24-GFP fusion protein. Chlorophyll: chlorophyll red autofluorescence reveals the chloroplasts; p24-GFP: fluorescence of the p24-GFP protein is detected in speckles in the chloroplasts; Syto85: The dye Syto85 reveals the presence of the DNA in the chloroplast. The green and blue spots can be superimposed, indicating that p24-GFP co-localizes with DNA in those cells.

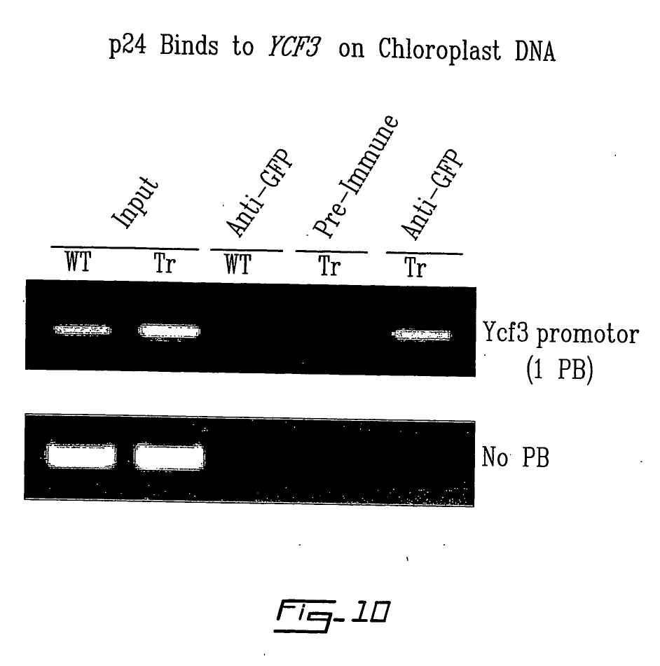

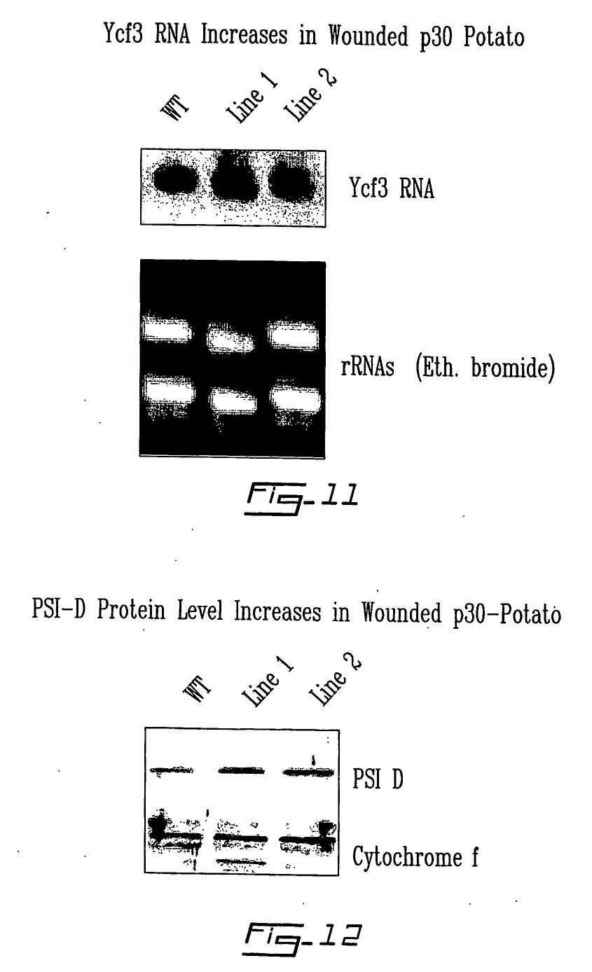

Results indicate that the level of YCF3 transcripts is indeed increased in transgenic potato plants overexpressing p24 (FIGS. 10 and 11). In FIG. 10, leaves of transgenic tobacco plants overexpressing a p24-GFP fusion proteins were treated with formaldehyde to cross-link proteins to DNA. Chloroplasts were isolated, DNA cleaved and p24-GFP immunoprecipitated with an anti-GFP antibody. DNA in the immunoprecipitated material was amplified with Taq polymerase using primers specific for the Ycf3 promoter (top panel), or a region of the chloroplast genome devoid of the PB element (bottom panel). Input: amplification before immunoprecipitation; Pre-immune: immunoprecipitation with a pre-immune antiserum; WT: untransformed tobacco; Tr: p24-GFP transgenic tobacco plants. FIG. 11 illustrates a northern blot (top panel) showing an increase of Ycf3 RNA in two independant transgenic lines of potato overexpressing the p30 gene. The bottom panel of FIG. 11 shows the ethidium bromide staining of ribosomal RNA in the RNA samples used to do the Northern blot.

As YCF3 is known to stabilize proteins involved in the formation of photosystem I, the accumulation of a protein of this photosystem, PSID, was measured. This protein is upregulated in p24 overexpressing plants (FIG. 12), therefore indicating that p24 affects expression of photosynthesis genes in plants. FIG. 12 illustrates western blot showing the increased accumulation of the PSID protein in two independant lines of potato overexpressing the p30 gene. The bottom panel of FIG. 12 shows that the level of cytochrome f is less affected in those plants. WT: Wild type plants.

The present invention is not intended to be limited only to peptide sequences for the transcriptional activators disclosed (either wild-type or mutant), but is intended to also include nucleic acid sequences which code for such transcriptional activator peptide sequences. The nucleic acid sequences can be recombinant nucleic acid molecules and may include DNA and RNA. Cloning and propagation of these nucleic acid molecules can be achieved by techniques commonly known and used in the art, such as by incorporating the nucleic acid molecules into vectors which can be transformed and/or transfected into bacterial or other host systems for maintenance and propagation thereof.

Expression of such peptides in a transformed host cell (for example, such as in a bacterial, fungal or plant cell) can be achieved by techniques commonly known and used in the art. For example, a recombinant nucleic acid molecule can be operatively linked to an expression control sequence in an expression vector. The expression vector can then be used to transform a host cell using techniques commonly known and used in the art, such as for example, Agrobacterium mediated transformation. The transformed host may be, for example, a single cell, or a callus of cells or plant produced by culturing the cell in vitro. Expression of the peptide in the transformed host can then be achieved by the appropriate measures, for example, by culturing the cell in an appropriate cell culture medium in vitro under conditions that provide for expression of the recombinant nucleic acid molecule by the cell, or by the application of an appropriate inducer.

Such transformed hosts can be used to produce large quantities of such peptides which can be isolated and purified from such transformed hosts. A vector transformed into a host cell may remain separate from the genome of the host or it may become integrated within the genome of the host. Transformed hosts are considered to be transgenic. Localization of the peptide may not be restricted to the nucleus.

A transgenic plant possessing a nucleic acid as discussed above and expressing the peptide coded by the nucleic acid would display increased pathogen resistance over a non-transgenic plant;

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A peptide which confers increased pathogen resistance upon a plant expressing said peptide, said peptide having a sequence selected from the group consisting of SEQ ID NO:1, SEQ ID NO:2, SEQ ID NO:3, and SEQ ID NO:4 mutated at a position selected from the group consisting of Glu271, Trp272 and residues between and including Pro123 to Gly128, an ortholog thereof, a homolog thereof, a functionally active fragment thereof or a functionally active variant thereof.

2. A recombinant nucleic acid molecule comprising a sequence which codes for a peptide selected from the group consisting of SEQ ID NO:1, SEQ ID NO:2, SEQ ID NO:3 and SEQ ID NO:4 mutated at a position selected from the group consisting of Glu271, Trp272 and residues between and including Pro123 to Gly128, an ortholog thereof, a homolog thereof, a functionally active fragment thereof or a functionally active variant thereof.

3. The recombinant nucleic acid molecule of claim 2, wherein said nucleic acid is DNA.

4. A vector containing the recombinant nucleic acid molecule of claim 2.

5. The recombinant nucleic acid molecule of claim 3, wherein said DNA sequence is operatively linked to an expression control sequence.

6. An expression vector containing the recombinant DNA molecule of claim 5.

7. A method of expressing a recombinant nucleic acid molecule in a cell containing the expression vector of claim 6, comprising culturing the cell in an appropriate cell culture medium under conditions that provide for expression of the recombinant DNA molecule by the cell.

8. The method of claim 7, further comprising the step of purifying a recombinant product of the expression of the recombinant DNA molecule.

9. A cell transformed with the recombinant DNA molecule of claim 2.

10. The cell of claim 9, wherein said recombinant DNA molecule is integrated in the genome of said cell.

11. The cell of claim 9, wherein said cell is a plant cell.

12. A cell expressing the peptide of claim 1.

13. A plant comprising the cell of claim 11 or 12.

14. A transgenic plant expressing the peptide of claim 1.

15. A transgenic plant comprising the recombinant nucleic acid molecule of claim 2.

16. The transgenic plant of claim 15, wherein said recombinant nucleic acid is integrated into the genome of said cell.

17. A method of increasing pathogen resistance in a plant comprising the steps of: (a) introducing into a cell of said plant a recombinant nucleic acid molecule as defined in claim 2; and (b) expressing said recombinant nucleic acid molecule in said cell.

18. A method of increasing pathogen resistance in a plant comprising the steps of: (a) mutating a nucleic acid sequence which codes for p24; and (b) expressing said nucleic acid sequence in said plant, wherein said mutating results in an amino acid substitution in said p24 which increases DNA binding affinity of PBF-2 for an elicitor response element (ERE).

19. The method of claim 18, wherein said amino acid substitution replaces Pro125 with nothing or a different amino acid.

20. The method of claim 18, wherein said amino acid substitution is Pro125 to Leu.

21. The method of claim 18, wherein said amino acid substitution replaces Trp272 with nothing or a different amino acid.

22. The method of claim 18, wherein said amino acid substitution is Trp272 to Ala.

23. The method of claim 18, wherein said amino acid substitution replaces Glu271 with nothing or a different amino acid.

24. The method of claim 18, wherein said amino acid substitution is Glu271 to any non-acidic amino acid.

25. The method of claim 18, wherein said ERE regulates expression of a pathogenesis-related (PR) gene.

26. The method of claim 25, wherein said PR gene is a PR-10 gene.

27. The method of claim 26, wherein said PR gene is PR-10a.

28. The method of claim 18, wherein the step of mutating a nucleic acid sequence is effected by a chemical mutagen, radiation, natural mutation or a recombinant DNA technique.

29. The method of claim 28, wherein said recombinant DNA technique is site-directed mutagenesis.

30. A method of increasing pathogen resistance in a plant comprising increasing DNA binding affinity of PBF-2 for an elicitor response element (ERE) of a pathogenesis-related (PR) gene.

31. The method of claim 30, wherein increasing DNA binding affinity of PBF-2 for an ERE comprises mutating a C-terminal negative autoregulatory domain of p24, wherein said C-terminal autoregulatory domain inhibits PBF-2 DNA binding and wherein said mutating decreases negative autoregulation of said domain.

32. The method of claim 31, wherein said mutating comprises an amino acid substitution in p24.

33. The method of claim 32, wherein said amino acid substitution replaces Pro125 with nothing or a different amino acid.

34. The method of claim 32, wherein said amino acid substitution is Pro125 to Leu.

35. The method of claim 32, wherein said amino acid substitution replaces Trp272 with nothing or a different amino acid.

36. The method of claim 32, wherein said amino acid substitution replaces Trp272 with Ala.

37. The method of claim 32, wherein said amino acid substitution replaces Glu271 with nothing or a different amino acid.

38. The method of claim 32, wherein said amino acid substitution replaces Glu271 with any non-acidic amino acid.

39. The method of claim 30, wherein said mutating a C-terminal negative autoregulatory domain is effected by a chemical mutagen, radiation, natural mutation or a recombinant DNA technique.

40. The method of claim 39, wherein said recombinant DNA technique is site-directed mutagenesis.

41. The method of claim 18, wherein said amino acid substitution replaces a residue between and including Pro123 to Gly128 with nothing or a different amino acid.

42. The method of claim 32, wherein said amino acid substitution replaces a residue between and including Pro123 to Gly128 with nothing or a different amino acid.

43. A method of increasing pathogen resistance in a plant comprising the step of overexpressing a nucleic acid coding for AtWhy1, StWhy1, an ortholog thereof or an analog thereof.

44. A method of increasing pathogen resistance in a plant comprising the step of overexpressing a pathogenesis-related (PR) gene.

45. The method of claim 44, wherein said PR gene is a PR-10 gene.

46. The method of claim 44, wherein said PR gene is PR-10a.
